(19) United States
(12) Patent Application Publication
(10) Pub. No.: US 2026/0090867 A1
(43) Pub. Date: Apr. 2, 2026

(54) Oral Irrigator Pump Body And An Oral Irrigator

(71) Applicant: LEI YU, Huangshi City (CN)

(72) Inventor: LEI YU, Huangshi City (CN)

(21) Appl. No.: 18/902,875

(22) Filed: Sep. 30, 2024

(51) Int. Cl. A61C 17/02; A61M 3/02

(52) U.S. Cl. CPC A61C 17/0202; A61M 3/0258; A61M 3/0279

(57) ABSTRACT

An oral irrigator pump body and an oral irrigator, including a bracket, a power device and a pump body structure connected thereto are provided in the bracket; the output end of the power device is provided with a transmission structure for driving the piston assembly to reciprocate in the pump cylinder, the transmission structure includes a driving wheel, a driven wheel, the first transmission wheel meshed with the driving wheel, the second transmission wheel meshed with the driven wheel, and a driving rod; an optical axis is fixedly provided on the first transmission wheel, and the second transmission wheel is provided on the optical axis and rotates synchronously with the first driven wheel.

### FIELD OF THE INVENTION

The present invention relates to the technical field of oral irrigators, and in particular to an oral irrigator pump body and an oral irrigator.

### BACKGROUND OF THE INVENTION

The oral irrigator (also called a dental water jet, water flosser) is an oral cleaning and health care device that uses a stream of high-pressure pulsating water generated by a motor intended to remove dental plaque and food residues between teeth and below the gum line, thereby achieving the effect of teeth cleaning and maintaining oral hygiene. The working principle of the oral irrigator is that the motor outputs torque, and drives the piston to make high-speed reciprocating motion in the pump through the transmission mechanism, which will cause intermittent changes in the internal volume of the pump, forming a negative pressure to suck the water in the water tank into the pump, and then the water is sprayed out from the nozzle with a strong water flow, aiming at the teeth to rinse and complete the cleaning function. Compared with traditional toothbrushes, the strong pulsed water flow of the oral irrigator can penetrate into the gaps between teeth, remove dirt that the toothbrush cannot reach, and ensure dental hygiene.

The transmission structure of the oral irrigator in the prior art is usually that the output shaft of motor drives the pinion gear, and then drives the large disc-shaped gear to rotate at high speed through gear meshing. The large gear drives the piston to reciprocate in the pump through the connecting rod mechanism, so that the volume of the inner cavity of the pump can be changed, and the functions of water pumping and spraying can be realized. However, this structure has certain defects. For example, the structure of the pinion gear driving the large disc-shaped gear occupies a large mechanical space, resulting in too big pump body size and size of the entire oral irrigator, which is not convenient for users to carry and use. When the large disc-shaped gear rotates at high speed, it will generate a lot of noise and body vibration, which seriously affects the users' experience and easily makes users mistakenly think that the product is faulty. A Chinese patent (Patent No.: ZL201921881283.X) achieves a certain noise reduction effect by setting sound-absorbing cotton, but the noise of the whole machine is still relatively loud. Due to the loud noise of the oral irrigator, it will bring a bad experience to users during use, and users can easily mistakenly think that the product is faulty and file complaints.

This traditional structure is difficult to meet the comfort requirements of users, so it is urgent to redesign and optimize the transmission mechanism to make the pump body of the oral irrigator realize miniaturization and low noise, thereby truly improving the user experience. The present invention has taken many improvement measures in the structural design of the pump body. For example, the two-part bracket structure makes the pump body easier for assembly and disassembly, the multi-stage gear meshing transmission makes the noise reduced, and the double-layer seal between the piston and the pump body makes the friction noise reduced during operation, etc. Through the comprehensive application of these structural design points, the working noise of this product is greatly reduced, which brings a more comfortable use experience to users and greatly improves the product quality. In general, the oral irrigator pump body of this application has made a breakthrough in structural design, and its performance are significantly higher than those of existing similar products.

In summary, the transmission structure of the current oral irrigator has problems such as large size, loud noise, and inconvenient to carry, which cannot meet the comfort requirements of users. It is urgent to comprehensively optimize the transmission structure to achieve miniaturization and low noise, so as to improve the user experience.

### SUMMARY OF THE DISCLOSURE

The present invention provides an oral irrigator pump body and an oral irrigator, aiming to solve the problem of loud noise in the oral irrigator during operation in the prior arts.

The present invention provides the following technical solutions:
An oral irrigator pump body includes a bracket, a power device and a pump body structure connected thereto are arranged inside the bracket; the pump body structure includes a pump cylinder and a piston assembly provided in the pump cylinder; a water outlet installation counterbore is arranged at the end of the pump cylinder, and a water inlet installation counterbore is arranged on the side wall of the pump cylinder;
The output end of the power device is provided with a transmission structure for driving the piston assembly to reciprocate in the pump cylinder. The transmission structure includes a driving wheel, a driven wheel, the first transmission wheel meshed with the driving wheel, the second transmission wheel meshed with the driven wheel, and a driving rod. The optical axis is fixedly provided on the first transmission wheel, the second transmission wheel is provided on the optical axis and rotates synchronously with the first driven wheel, one end of the driving rod is sleeved on the driven wheel, and the other end of the driving rod is fixedly connected to the piston assembly.

Preferably, the bracket includes the first bracket and the second bracket that are detachably connected, and a mounting cavity that is matched with the transmission structure is formed in the first bracket.

Preferably, the first transmission wheel and the second transmission wheel are integrally formed, a mounting hole for installing the optical axis is provided in the mounting cavity, one end of the optical axis passes through the first transmission wheel and the second transmission wheel in sequence and is fixed in the mounting hole, and the other end of the optical axis is fixed to the second bracket.

Preferably, the first transmission wheel and the optical axis are integrally formed, and the optical axis cooperates with the second transmission wheel.

Preferably, the piston assembly includes a piston head and a movably connected piston rod, the piston head is provided with a first leak-proof sealing ring, the side wall of the piston rod is provided with a mounting ring groove, a second leak-proof sealing ring is provided in the mounting ring groove, and the first leak-proof sealing ring and the second leak-proof sealing ring are airtightly slidably connected to the pump cylinder.

Preferably, the piston head and one end of the pump cylinder corresponding to the water outlet installation counterbore form the first movement cavity, the piston head and one end of the pump cylinder corresponding to the second sealing ring form the second movement cavity, and the first movement cavity is connected to the water inlet installation counterbore and the water outlet installation counterbore.

Preferably, a water leakage hole is provided on the side wall of the pump cylinder, and the second movement cavity is always connected to the water leakage hole during the movement.

Preferably, the water outlet installation counterbore is detachably or fixedly connected with a water outlet mechanism, the water outlet mechanism includes a water outlet connector and a duckbill valve arranged inside the water outlet connector, the water inlet installation counterbore is detachably fixedly connected with a water inlet mechanism; the water inlet mechanism includes a water inlet joint and a one-way valve plate provided inside the water inlet joint, and the pump cylinder controls the opening and closing of the one-way valve plate by the reciprocating motion of the piston assembly in the pump cylinder.

In addition, an oral irrigator includes the oral irrigator pump body and a nozzle assembly detachably or fixedly connected thereto according to any one of technical solutions above-mentioned.

Furthermore, the nozzle assembly includes a nozzle base and a nozzle head detachably or fixedly connected thereto.

The beneficial effects of the present invention are as follows: firstly, the gear rotation speed is reduced through the transmission structure of multi-stage gear meshing, and the noise generated by the impact of gear meshing can be reduced; secondly, the double-layer seal of the piston and the pump cylinder improves the sealing performance, and the friction noise during operation can be reduced. Through the mutual cooperation of these design points, the noise level of the oral irrigator pump body can be greatly reduced when it is working, thereby improving the user experience.

### BRIEF DESCRIPTION OF THE DRAWINGS

Other features, objects and advantages of the present invention will be more clearly and completely by the detailed description of the non-limiting embodiments with reference to the attached drawings. Obviously, the described embodiments are only part of the embodiments of the present invention, but not all of them. Based on the embodiments of the present invention, all other embodiments obtained by ordinary technicians in the field without creative labor are within the protection scope of the present invention.

FIG. 1 is a schematic view of an explosion structure of the first embodiment in the present invention.

FIG. 2 is a perspective view of the three-dimensional structure of the fourth embodiment in the present invention.

FIG. 3 is a schematic structural view of the nozzle assembly in the present invention.

FIG. 4 is a cross-sectional view of the fourth embodiment in the present invention.

### DETAILED DESCRIPTION OF THE PREFERRED EMBODIMENTS

In the present invention, the use of orientation words such as “outside”, “inside”, “left”, “right” are only for the convenience of description, rather than indicating or implying the specific orientation, therefore it should not be construed as being limited to the description of the following embodiments. In addition, it should be noted that the terms “first”, “second”, “third” and the like in the description and claims of the present invention and the above drawings are used to distinguish similar objects, which are not necessarily used to describe the specific order or sequence. In the description of the present invention, “multiple” means at least two, such as two, three, etc., unless otherwise specifically limited. It is to be understood that the data so used are interchangeable under appropriate circumstances in order to describe the embodiments of the invention herein.

Unless otherwise stated, it should be noted that the terms “installed” and “connected” should be understood broadly. For example, “connected” could be fixed connection, detachable connection, integral connection, mechanical connection, electrical connection, direct connection, indirect connection through the intermediate structure, or internal connection between two elements. For those of ordinary skill in this field, the specific meanings of the above terms in the present invention can be understood in specific situations.

In the present invention, unless otherwise stated and limited, the first feature being “above” or “below” the second feature may mean that the first and second features are in direct contact, or the first feature and second features are in indirect contact through an intermediate medium. Moreover, the first feature being “above” the second feature may mean that the first feature is directly above or obliquely above the second feature, or simply means that the first feature is higher in level than the second feature. The first feature being “below” the second feature may mean that the first feature is directly below or obliquely below the second feature, or simply means that the first feature is lower in level than the second feature.

As a popular oral hygiene and health care device, the working principle of the oral irrigator is that the motor outputs torque, and drives the piston of the water pump to make high-speed reciprocating motion in the pump body through the transmission mechanism, which will cause intermittent changes in the internal volume of the pump body, forming a negative pressure to pump the water in the water tank into the pump body, and then it is converted into positive pressure to spray the water from the nozzle with a strong water flow, and remove food residues, dental plaque and other dirt on the tooth surface through mechanical impact, so as to achieve the purpose of cleaning and hygiene. Compared with traditional toothbrushes, the impact force and cleaning depth of pulse water flow of the oral irrigator are better, and it can penetrate deep into the teeth to remove dirt that the toothbrush cannot reach.

The core of the current oral irrigator products, namely the pump body, mainly includes three parts: a motor, a transmission structure and a water pump. The transmission structure of many products adopts a method in which the pinion gear drives the large disc-shaped gear, and then drives the piston to reciprocate at high speed in the pump body through a connecting rod mechanism. This structure can indeed achieve the functions of water pumping and spraying, but it also has certain defects, including but not limited to large size, loud noise, inconvenience in carrying and using, etc., and cannot provide users with a comfortable and smooth use experience. Specifically, when the large disc rotates at high speed, it will generate very loud operating noise and vibration, which seriously affects the comfort of use, and users can easily mistakenly think that the product is faulty. Some products have achieved certain effects by setting sound absorption and other measures to reduce noise, but the overall noise is still loud, which will make users feel uncomfortable and have a bad impression of the products.

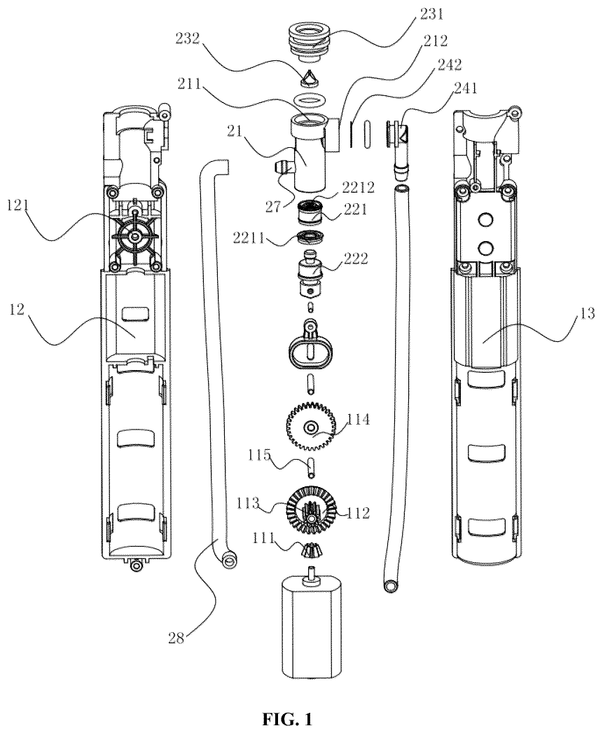

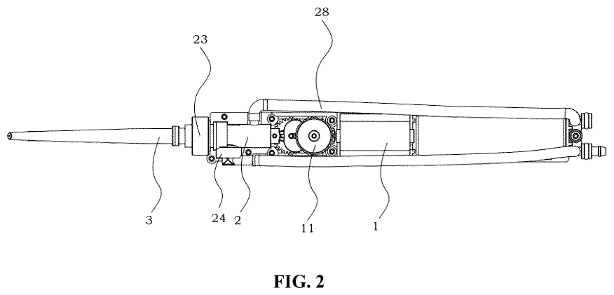

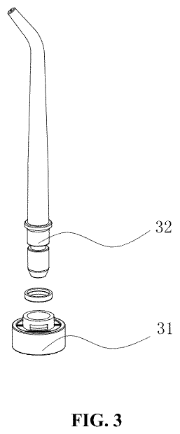

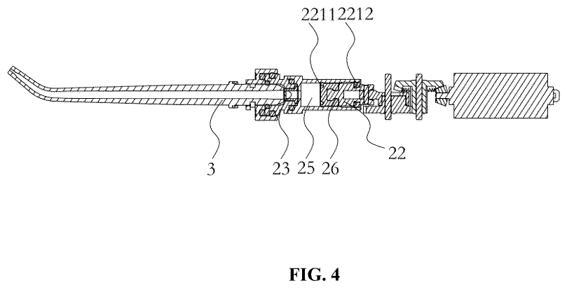

Other features, objects and advantages of the present invention will be more clearly and completely by the detailed description of the non-limiting embodiments with reference to the attached drawings (FIG. 1 to FIG. 4).

Embodiment 1: The first embodiment provides an oral irrigator pump body, including a bracket 1, a power device and a pump body structure 2 connected thereto are provided inside the bracket 1, and the power device is electrically connected to the power source. In this embodiment, the power device is a small electric motor, and the electric motor is electrically connected to the positive and negative poles of the rechargeable battery through the wiring terminal, and the rechargeable battery provides working current for the electric motor. The pump body structure 2 includes a cylindrical pump cylinder 21 and a piston assembly 22 provided inside the pump cylinder 21, the end of the pump cylinder 21 is provided with a water outlet installation counterbore 211 for installing the water outlet mechanism 23, and the side wall of the pump cylinder 21 is provided with a water inlet installation counterbore 212 for installing the water inlet mechanism 24.

The piston assembly 22 is composed of two major parts: a piston head 221 at its front end and a piston rod 222 movably connected thereto. An annular first leak-proof sealing ring 2211 made of rubber material is fixedly provided on the outer circumferential surface of the piston head 221 to increase the contact pressure with the inner wall of the pump cylinder 21. At the same time, a circle of annular mounting grooves is provided in the middle section of the side wall of the piston rod 222. A second leak-proof sealing ring 2212 is embedded in the groove and the second leak-proof sealing ring 2212 is made of rubber material. The outermost diameter of the second leak-proof sealing ring 2212 is slightly larger than the inner diameter of the pump cylinder 21 to ensure airtight performance. When the power device drives the transmission mechanism to work, it will push the piston assembly 22 to slide back and forth in the pump cylinder 21. At this time, the first leak-proof sealing ring 2211 and the second leak-proof sealing ring 2212 always keep in contact with the inner wall of the pump cylinder 21 and slide in an airtight friction state to prevent the liquid in the pump cylinder 21 from leaking.

The piston head 221 divides the internal space of the pump cylinder 21 into two independent cavities. The piston head 221 and one end of the pump cylinder 21 corresponding to the water outlet installation counterbore 211 form a first movement cavity 25, and the piston head 221 and one end of the pump cylinder 21 corresponding to the second sealing ring form a second movement cavity 26. The water inlet installation counterbore 212 and the water outlet installation counterbore 211 are both connected to the first movement cavity 25, and the volume of the first movement cavity 25 and the second movement cavity 26 changes with the reciprocating motion of the piston. Considering that during long-term use, a small amount of water may enter the second movement cavity 26 through the matching gap between the piston rod and the pump cylinder 21. If it cannot be discharged in time, the accumulated water will affect the reciprocating motion of the piston and may even damage the sealing ring. Therefore, a water leakage hole 27 is opened on the side wall of the pump cylinder 21, and it is always connected to the second movement cavity 26. A water leakage silicone tube 28 is connected to the water leakage hole 27, a water leakage tube joint is connected to the end of the water leakage silicone tube 28, and the liquid that penetrates into the second movement cavity 26 is discharged outward through the water leakage hole 27 and the water leakage silicone tube 28.

The water outlet mechanism 23 is detachably or fixedly installed at the water outlet installation counterbore 211, the water outlet mechanism 23 includes a water outlet connector 231 and a duckbill valve 232 which is provided inside the water outlet connector 231 and only allows water to flow in one direction. The water inlet mechanism 24 is detachably fixedly installed at the water inlet installation counterbore 212, and the water inlet mechanism 24 includes a water inlet joint 241 and a one-way valve plate 242 which is provided inside the water inlet joint 241. The pump cylinder 21 controls the opening and closing of the one-way valve plate 242 by reciprocating motion of the piston assembly 22 in the pump cylinder 21.

The output shaft of the electric motor drives the piston assembly 22 to reciprocate in the pump cylinder 21 through the transmission structure 11. The transmission structure 11 specifically includes a driving wheel 111 provided on the output shaft of the electric motor, a first transmission wheel 112 meshing with the driving wheel 111, an optical axis 115 fixedly provided on the first transmission wheel 112, a second transmission wheel 113 provided on the optical axis 115 and rotated synchronously with the first transmission wheel 112, the second transmission wheel 113 is meshed with the driven wheel 114, one end of the driving rod is sleeved on the driven wheel 114, and the other end of the driving rod is fixedly connected to the piston rod 222.

When the power device is started and the piston assembly 22 is driven to reciprocate through the transmission structure 11, the volume of the first movement cavity 25 changes periodically. When the volume of the first movement cavity 25 increases, the one-way valve plate 242 opens, and the liquid from external water tank is pumped into the pump cylinder 21; when the volume of the first movement cavity 25 decreases, the liquid is pressed out of the pump cylinder 21 through the duckbill valve 232.

### Embodiment 2

The difference between the second embodiment and the first embodiment is that the bracket 1 is detachably assembled by connecting the first bracket 12 and the second bracket 13 by bolt or buckle connection. The first bracket 12 is provided with an installation cavity 121 matching the size of the transmission structure 11. The installation cavity 121 is provided with a circular mounting hole for mounting the optical axis 115 in the transmission structure 11. The driving wheel 111 is fixed on the output shaft of the electric motor, and the first transmission wheel 112 is meshed with the driving wheel 111 and fixed at one end of the optical axis 115. The second transmission wheel 113 and the first transmission wheel 112 are integrally formed by injection molding, and the optical axis 115 is sequentially passed through the axial holes of the second transmission wheel 113 and the first transmission wheel 112 and installed in the mounting hole of the optical axis 115, the driven wheel 114 is gear-meshed with the second transmission wheel 113, and one end of the connecting rod is hinged in the eccentric hole of the driven wheel 114. Specially during assembling, the first transmission wheel 112 and the second transmission wheel 113 are first passed through the optical axis 115 in sequence, and then the left end of the optical axis 115 is inserted into the mounting hole of the first bracket 12. The right end of the optical axis 115 passes through and is fixed in the second bracket 13. When the electric motor is started, its output shaft drives the driving wheel 111 to rotate, the driving wheel 111 drives the first transmission wheel 112 and the second transmission wheel 113 to rotate synchronously, the second transmission wheel 113 drives the driven wheel 114 to rotate, and the driven wheel 114 drives the piston assembly 22 to reciprocate in the pump cylinder 21 through the connecting rod to complete the water pumping and spraying functions. The multi-stage gear transmission structure 11, which drives the large gear with the small gear, plays a role of deceleration, and the gear meshing is more stable and precise, which can greatly reduce the vibration and noise of the gear during high-speed transmission.

### Embodiment 3

The difference between the third embodiment and the second embodiment is that the optical axis 115 is integrally formed with the first transmission wheel 112, and the first transmission wheel 112 is driven to rotate by the driving wheel 111, thereby driving the integrally formed optical axis 115 to rotate. The second transmission wheel 113 cooperates with the optical axis 115, and rotates synchronously under the drive of the optical axis 115. The driven wheel 114 rotates in mesh with the second transmission wheel 113, drives the connecting rod through the eccentric wheel structure, and then pushes the piston assembly 22 to reciprocate in the pump body to complete the work of the water pumping and spraying.

### Embodiment 4

The fourth embodiment provides an oral irrigator, comprising an oral irrigator pump body and a nozzle assembly 3 according to any of the above embodiments. The nozzle assembly 3 comprises a nozzle base 31 and a nozzle head 32, which are detachably connected by threads. The nozzle base 31 is made of metal and has a corrugated water flow channel inside, which matches the shape of the water outlet installation counterbore 211 of the oral irrigator pump body. The internal thread is provided at one end of the nozzle base 31 away from the pump body, for installing the nozzle head 32. A water outlet hole is provided on one side of the nozzle head 32 away from the nozzle base 31. A nozzle decorative ring is also provided on the outside of the nozzle head 32.

When the oral irrigator is working, the liquid enters the pump cylinder 21 under the suction of the oral irrigator pump body. The piston inside the pump cylinder 21 reciprocates at high speed under the drive of the power device and the transmission device, and is spraying through the nozzle assembly 3 after accumulating energy to form a high-pressure water flow. After the rectification and diameter reduction of the corrugated flow channel in the nozzle base 31, and the focusing of the nozzle head 32, the diameter of the high-pressure water column is rapidly reduced, the pressure and speed are greatly increased, thereby obtaining an excellent flushing effect. The nozzle assembly 3 adopts a detachable design, which is easy to clean and replace. At the same time, the corrugated inner cavity design can minimize water flow turbulence to the greatest extent, reduce fluid resistance, and improve water discharge efficiency.

Hereinafter, embodiments of the present invention have been described in detail with reference to the accompanying drawings. While the description above refers to the particular embodiments of the present invention, it will be understood that many modifications may be made without departing from the spirit thereof. Any equivalent replacement or modification would fall within the protection scope of the present invention.

What is claimed is:

1. An oral irrigator pump body comprising a bracket, a power device and a pump body structure connected thereto are provided inside the bracket; the pump body structure includes a pump cylinder and a piston assembly provided inside the pump cylinder; a water outlet installation counterbore is provided at the end of the pump cylinder, and a water inlet installation counterbore is provided on the side wall of the pump cylinder; an output end of the power device is provided with a transmission structure for driving the piston assembly to reciprocate in the pump cylinder. The transmission structure includes a driving wheel, a driven wheel, the first transmission wheel meshed with the driving wheel, the second transmission wheel meshed with the driven wheel, and a driving rod,
wherein an optical axis is fixedly provided on the first transmission wheel, the second transmission wheel is provided on the optical axis and rotates synchronously with the first driven wheel, one end of the driving rod is sleeved on the driven wheel, and the other end of the driving rod is fixedly connected to the piston assembly.

2. An oral irrigator pump body according to claim 1, wherein the bracket includes a first bracket and a second bracket that are detachably connected, and a mounting cavity that is matched with the transmission structure is formed in the first bracket.

3. An oral irrigator pump body according to claim 2, wherein the first transmission wheel and the second transmission wheel are integrally formed, a mounting hole for installing the optical axis is provided in the mounting cavity, one end of the optical axis passes through the first transmission wheel and the second transmission wheel in sequence and is fixed in the mounting hole, and the other end of the optical axis is fixed to the second bracket.

4. An oral irrigator pump body according to claim 2, wherein the first transmission wheel and the optical axis are integrally formed, and the optical axis cooperates with the second transmission wheel.

5. An oral irrigator pump body according to claim 1, wherein the piston assembly includes a piston head and a movably connected piston rod, the piston head is provided with a first leak-proof sealing ring, the side wall of the piston rod is provided with a mounting ring groove, the second leak-proof sealing ring is provided in the mounting ring groove, and the first leak-proof sealing ring and the second leak-proof sealing ring are airtightly slidably connected to the pump cylinder.

6. An oral irrigator pump body according to claim 5, wherein the piston head and one end of the pump cylinder corresponding to the water outlet installation counterbore form the first movement cavity, the piston head and one end of the pump cylinder corresponding to the second sealing ring form the second movement cavity, and the first movement cavity is connected to the water inlet installation counterbore and the water outlet installation counterbore.

7. An oral irrigator pump body according to claim 6, wherein a water leakage hole is provided on the side wall of the pump cylinder, and the second movement cavity is always connected to the water leakage hole during the movement.

8. An oral irrigator pump body according to claim 7, wherein the water outlet installation counterbore is detachably fixedly connected with a water outlet mechanism, the water outlet mechanism includes a water outlet connector and a duckbill valve provided inside the water outlet connector, the water inlet installation counterbore is detachably or fixedly connected with a water inlet mechanism; the water inlet mechanism includes a water inlet joint and a one-way valve plate provided inside the water inlet joint, and the pump cylinder controls the opening and closing of the one-way valve plate by the reciprocating motion of the piston assembly in the pump cylinder.

9. An oral irrigator pump body according to any one of claim 1, further comprising an oral irrigator pump body and a nozzle assembly or detachably fixedly connected thereto.

10. An oral irrigator pump body according to claim 9, wherein the nozzle assembly includes a nozzle base and a detachably or fixedly connected nozzle head.
